(19) United States
(12) Patent Application Publication
(10) Pub. No.: US 2016/0151149 A1
(43) Pub. Date: Jun. 2, 2016

(54) VISION CORRECTION SYSTEM

(71) Applicant: University of Utah Research Foundation, Salt Lake City, UT (US)

(72) Inventor: Randall J. Olson, Salt Lake City, UT (US)

(21) Appl. No.: 15/015,925

(22) Filed: Feb. 4, 2016

Related U.S. Application Data
(63) Continuation of application No. 13/275,175, filed on Oct. 17, 2011.
(60) Provisional application No. 61/394,327, filed on Oct. 18, 2010.

(51) Int. Cl. A61F 2/16

(52) U.S. Cl. CPC A61F 2/1602; A61F 2/161; A61F 2/1648

(57) ABSTRACT

A vision correction system comprising an intraocular lens having a lens body and one or more haptics is provided. The lens body may be configured to be positioned posteriorly to an iris of an eye and may have a convex anterior surface, a concave posterior surface, and a circumferential edge having a rounded anterior portion and a rounded posterior portion. Haptics may extend at an anterior angle from the lens body and be configured to contact the ciliary sulcus of an eye. The haptics may secure the intraocular lens in the eye in a relatively fixed position and prevent rotation of the lens over time.

### RELATED APPLICATION

The present application claims the benefit of U.S. Provisional Patent Application Ser. No. 61/394,327, filed Oct. 18, 2010, which is incorporated herein by reference in its entirety.

### FIELD

The present invention relates generally to vision correction, and more particularly to an intraocular lens, such as a piggy-back lens, which may supplement an intraocular lens (IOL) implanted in an eye.

### BACKGROUND

An intraocular lens may be implanted in an eye to replace a natural crystalline lens that has become cloudy by a cataract and/or may be implanted as part of refractive surgery to adjust the optical power of the eye.

### SUMMARY

After cataract or refractive surgery, there is often a residual refractive error and/or other error that leaves the patient unsatisfied with the results. Further, patients are increasingly demanding a perfect refractive result after cataract or refractive surgery. Therefore, there is a need for a piggy-back lens that supplements an intraocular lens (IOL) implanted in an eye to correct for residual refractive error and/or other error, thereby providing better results.

In accordance with one aspect of the present invention, a piggy-back intraocular lens is disclosed. The piggy-back intraocular lens may comprise a lens body, at least a portion of which is transparent, the lens body configured to be positioned posteriorly to an iris of an eye and having a convex anterior surface, a concave posterior surface, and a circumferential surface at a circumference of the lens body. The piggy-back intraocular lens also may comprise one or more haptics extending from the lens body, the one or more haptics configured to fit in the ciliary sulcus of the eye when the lens is positioned posteriorly to the iris. The circumferential surface of the lens body may have a rounded anterior edge and a rounded posterior edge along at least a portion of the circumference.

In one aspect, a method of correcting a residual refractive error in an eye after implantation of a first intraocular lens in the eye is disclosed. The method may comprise inserting a second intraocular lens into the eye. The second lens may have a transparent portion and may comprise a lens body having a convex anterior surface, a concave posterior surface, a circumferential surface and/or one or more haptics extending from the lens body. The method may also comprise positioning the lens body in the eye so that the posterior surface of the lens body contacts at least one of the anterior surface of the first intraocular lens and peripheral aspect of the anterior capsule. Additionally, the method may comprise the step of contacting outer portions of the one or more haptics with the ciliary sulcus of the eye to secure the second lens in a relatively fixed position in the eye.

Additional features and advantages of the invention will be set forth in the description below, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed. It will be further understood the numerous modifications may be made to the embodiments discussed in the detailed description without departing from the scope or spirit of the invention. Such modifications may include, but are not limited to, size, shape, materials and modifications regarding such are intended to fall within the scope of the invention unless expressly set forth to the contrary in the claims.

### BRIEF DESCRIPTION OF THE DRAWINGS

FIG. 1A shows a side view of a piggy-back lens according to an aspect of the present invention;

FIG. 1B shows a side view of the piggy-back lens and a blown-up, side view of an edge of the piggy-back lens according to an aspect of the present invention;

FIG. 2 shows a top view of the piggy-back lens according to an aspect of the present invention; and

FIG. 3 shows the piggy-back lens implanted in an eye to supplement an intraocular lens (IOL) according to an aspect of the present invention; and

FIG. 4 shows a side view of a piggy-back lens according to another aspect of the present invention;

It will be appreciated that the drawings are illustrative and not limiting of the scope of the invention which is defined by the appended claims. The embodiments shown accomplish various aspects and objects of the invention. It is appreciated that it may not be possible to clearly show each element and aspect of the invention in a single figure, and as such, multiple figures are presented to separately illustrate the various details or aspects of the invention in greater clarity. Similarly, not every embodiment or aspect need accomplish all advantages of the present invention and the invention is defined by the appended claims, rather than any particular embodiment or aspect set forth herein.

### DETAILED DESCRIPTION

The description of the invention is provided to enable a person of ordinary skill in the art to practice the various aspects of the invention described herein. While the present invention has been particularly described with reference to the various figures and embodiments, it should be understood that these are for illustration purposes only and should not be taken as limiting the scope of the invention.

One approach to correct for residual error after implantation of an IOL is LASIK surgery. While effective, the standard deviation of the results can be as great as the error the surgeon is trying to correct. Add to this the variation of the healing response, especially in older patients, and this approach may not be very accurate and may provide results which are inadequate for the patient. Also, LASIK surgery requires a laser and expertise that many cataract surgeons may not have. While all patients have symptoms of dry eyes after LASIK, which can be severe and persistent, older patients may be particularly prone to this and can leave many very dissatisfied with the results.

Another approach to correcting residual error after implantation may be to exchange the old IOL with a new IOL, which may require removal of the old IOL in the eye and placement of the new IOL. This may be difficult due to scarring of the IOL in the capsular tissue, and may have a complication rate greater than the original cataract surgery. Furthermore, the bag position can shift due to the surgery resulting in refractive error again after this procedure. Furthermore, the range of what is acceptable for IOL powers as marked can be enough to leave residual refractive error that is not acceptable.

Embodiments of the present invention provide piggy-back lenses that correct for residual errors of IOLs while avoiding one or more of the above-mention drawbacks of LASIK surgery and IOL exchange. A piggy-back lens may have fewer complications than the other approaches, and, because the underlying refractive error as well the biometry of the eye is well known, may be more accurate for the correction of refractive error. The piggy-back lens can be implanted in the eye through the original incision for the IOL. As a result, the complication rate is low and the procedure can be performed in several minutes.

It will be appreciated that, as use herein, the term piggy-back lens refers to a second lens which is placed in the eye in addition to the IOL. It is not meant to suggest a relative location between the two lenses, i.e. which lens is disposed in front of the other.

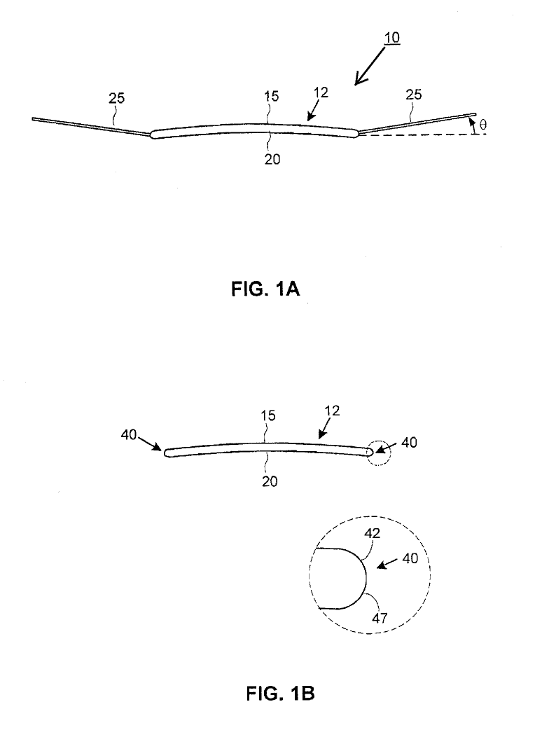

FIG. 1A shows a side view of a piggy-back lens 10 according to an aspect of the present invention. The piggy-back lens 10 may comprise or include a lens body 12 and two or more haptics 25 extending from the lens body 12. The lens body 12 may provide optical correction of residual error of an IOL implanted in an eye, and the haptics 25 may anchor the piggy-back lens 10 in the eye.

As shown in FIG. 1A, the haptics 25 may be anteriorly angled from the lens body 12 at an angle of θ, which may range, for example from about 5 to 10 degrees, though larger or small ranges may be indicated in some instances. Benefits of anteriorly angled haptics are discussed below.

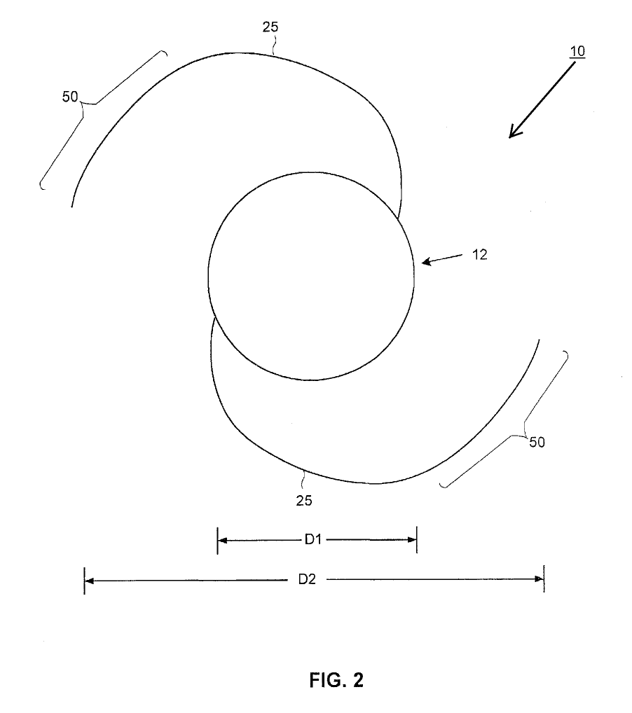

Referring to FIG. 2, the piggy-back lens 10 is shown to include the lens body 12 and two haptics 25. The lens body has a first diameter, D1, while the haptics 25 have a second diameter D2, which is greater than D1 and extends to the outside edge of the haptics. The haptic outside diameter D2 from the outside curve of one haptic 25 to the outside curve of the second haptic 25 may be wide enough to ensure good fixation in the ciliary sulcus even in large eyes. For example, the outside diameter D2 may be about 14.5 mm. However, other outside diameters D2 may also be used to fit different sized eyes.

As shown in FIG. 2, the haptics 25 may have broad ciliary sulcus contact portions 50 for contacting the ciliary sulcus when the piggy-back lens 10 is implanted in the eye. The broad contact between the ciliary sulcus and the haptics 25 may help minimize any compressive point tissue pressure necrosis and prevent rotation of the piggy-back lens 10 over time. The haptics 25 may have a relatively flat compression/tissue tension profile (gently curving from the lens body 12) for the same reason.

The haptics 25 may have thin (e.g., no more than 100 microns in AP thickness), polished and rounded edges to avoid iris damage or contact. Iris damage can result in pigment dispersion glaucoma, hemorrhage from the damaged iris, iritis (intraocular inflammation with all its consequences for eye health and vision), and glare due to the loss of iris light shielding over time. The haptics 25 can be made of any material. The haptics 25 may comprise separate pieces that are attached to the lens body 12 (e.g., three piece piggy-back lens 10) or may be integral with the lens body 12 (e.g., one piece piggy-back lens 10).

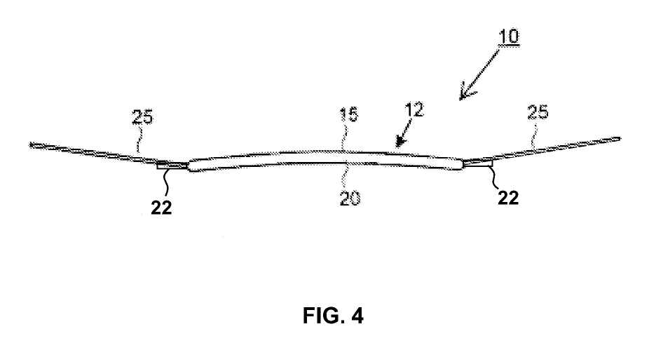

According to one aspect of the invention, as shown in FIG. 4, the lens body 12 of a three piece piggy-back lens may be comprised of a flange 22 extending from the lens body 12 for receiving the haptics 25. For example, the haptics 25 may be staked in or to the flange 22 to ensure that the haptics 25 remain securely attached to the lens body 12. Because a thin haptic 25 (e.g., no more than 100 microns in AP thickness) may be attachable to the lens body in such a manner, the likelihood that the haptics 25 will damage or contact the iris is substantially reduced and/or eliminated. It will be appreciated that a thicker haptic in a secondary lens may rub against the iris and cause damage thereto. The thinner haptic 25 associated with the flange 22 may reduce this risk.

Referring back to FIG. 1A, in one embodiment, the lens body 12 may have a convex anterior surface 15 and a concave posterior surface 20. The lens body 12 may be used to optically correct residual refractive error and/or other error (e.g., higher order aberrations or presbyopia) after an IOL has been implanted in the eye. In this embodiment, the curvature of the anterior surface 15, the curvature of the posterior surface 20 and/or the refractive index of lens body 12 may be chosen to correct residual refractive error for a particular patient. The residual error may be determined by performing an eye examine on the patient after the IOL has been implanted and/or other known techniques. In another embodiment, the lens body 12 may be shaped to have different optical powers in different meridians to correct, for example, astigmatism, higher order optical aberrations, etc.

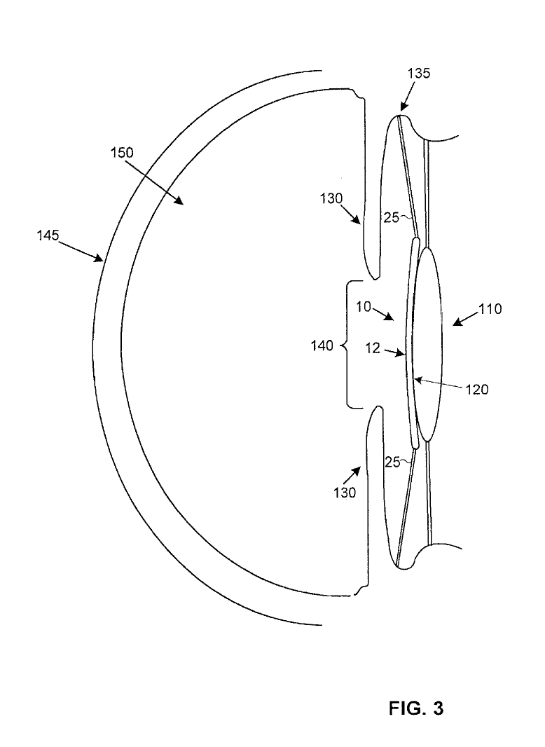

In one embodiment, the curvature of the posterior surface 20 may approximately match the curvature of the anterior surface of the IOL 110 so that the lens body 12 can be placed flush with the IOL 110 (shown in FIG. 3). This may allow the lens body 12 to hug the IOL 110 and wrap around the IOL 110.

As shown in FIG. 1A, the convex/concave shape may minimize the profile of the lens body 12. The thin profile may help the lens body 12 avoid contact with the posterior surface of the iris, and thus avoid the problems resulting from iris contact discussed above. The concave posterior surface 20 may also ensure centration of the piggy-back lens 10 on the anterior surface of the IOL 110 (shown in FIG. 3). In contrast, a piggy-back lens that has a convex posterior surface will tend to decenter because contact is at one point, with the natural tendency for that point to want to slide to a lower point with posterior pressure which is always applied. The concave posterior surface 20 may also provide broad optic to optic contact between the lens body 12 and the IOL 110 (shown in FIG. 3) so that point pressure between the optics will not result in some central optic flattening over time with resultant loss of refractive effect.

The lens body 12 may include one or more features to prevent Pseudophakic Dysphotopsia (PD). PD is a common problem after IOL insertion and may result in the presence of unwanted flashes, grey shadows and other photic images after cataract surgery. These images are common, often persistent, and a major complaint for patients who have had uncomplicated cataract surgery. PD is related to the optic size of the IOL (typically a larger IOL decreases the incidence of PD), optic edge treatment (a rounded edge may be preferred), refractive index of the optic (typically a higher refractive index may correlate with more severe PD), and the thinness of the material of the IOL (a thicker IOL may be better, so as to fill in more of the space between the optic and the iris).

In one embodiment, the lens body 12 may overlap the circumference of the IOL 110 to minimize any PD (shown in FIG. 3). This may be accomplished for most IOLs by making the optic diameter D1 (FIG. 2) between about 7 to 8 mm.

In one embodiment, the circumferential edge 40 of the lens body 12 may be smoothly rounded. As shown in the example in FIG. 1B, both the anterior portion 42 and the posterior portion 47 of the circumferential edge 40 may be rounded. In one embodiment, the circumferential edge 40 may have a semi-circular shape or other rounded shape. This may result in the least possible PD and also minimize any iris damage if contact with the iris posterior surface occurs, which should be infrequent.

In one embodiment, the optic material of the lens body 12 may have a refractive index which is likely to ameliorate PD and to be protective against intralenticular opacification (ILO). For example, the optic material may comprise a silicone material which generally has a low refractive index and is resistant to ILO. In addition, a silicon material may be least likely to be hydrophilic acrylic, which is most likely to result in ILO even with the piggy-back lens in the sulcus. In one embodiment, the optic material may have a refractive index of about 1.48 or less to prevent PD.

Thus, the lens body 12 may include one or more of the above features to treat PD including rounded edges and a low refractive index.

FIG. 3 shows an example of the piggy-back lens 10 implanted in the eye to supplement an IOL 110. FIG. 3 also shows the cornea 145, anterior chamber 150, iris 130 and ciliary sulcus 135 of the eye. The piggy-back lens 10 may be implanted through the same incision used to implant the IOL 110, and may be implanted during the same surgical procedure as the IOL 110 and/or at a later time. For example, the piggy-back lens 10 may be implanted post cataract surgery or refractive surgery where the patient is pseudophakic to correct residual refractive error and/or other error after the surgery.

In the example in FIG. 3, the IOL 110 may be implanted in the capsular bag and the piggy-back lens 110 may be implanted in the ciliary sulcus 135. Because the piggy-back lens 10 in this example is not implanted in the capsular bag, the piggy-back lens 10 can be exchanged with a new piggy-back lens 10 to correct for changing refractive error over time without scaring ocular tissue. Further ciliary sulcus fixation of the piggy-back lens 10 may avoid compressive forces that can rotate or decenter an IOL over time as well as prevent the problem of ILO, all of which can occur when both lenses are in the capsular bag.

As shown in FIG. 3, the haptics 25 may anchor the piggy-back lens 10 in the ciliary sulcus. As discussed above, the broad ciliary sulcus contact portions 50 of the piggy-back lens 10 (shown in FIG. 2) may provide broad contact between the ciliary sulcus 135 and the piggy-back lens 10 (the broad contact is perpendicular to the side view shown in FIG. 3). The broad contact may help achieve good centration, non-rotation and tissue gentleness.

As shown in FIG. 3, the anterior angle of the haptics 25 may move the lens body 12 toward the IOL 110 so that the posterior surface 20 (FIGS. 1A and 1B) of the lens body contacts the anterior surface of the IOL 110. This helps ensure that the lens body 12 lies flush with the anterior surface of the IOL 110, which improves refractive precision because the position of the lens body is more certain. Furthermore, the contact forces between the surfaces of the lens body 12 and the IOL may prevent rotation of the lens body 12, which may improve the stability of the piggy-back lens 10 over time. Additionally, the anterior angle of the haptics 25 may substantially prevent the piggy-back lens 10 from vaulting, i.e. will keep the lens body 12 away from the iris 130 to avoid iris contact and minimizing the risk that the lens body 12 will be captured by the pupil 140. Also, the convex/concave shape of the lens body may reduce the profile of the lens, which may further help avoid iris contact.

In one embodiment, the posterior surface 20 of the lens body 12 lies flush with the anterior surface 120 of the IOL 110. In this embodiment, at least about 25%, 50% or 75% of the posterior surface 20 of the lens body 12 may be in contact with the anterior surface 120 of the IOL 110 after implantation.

In another embodiment, the piggy-back lens 10 may be implanted such that the piggy-back lens 10 contacts residual and/or peripheral aspects of the anterior capsule. In this application of the invention, the central optic of the piggy-pack lens 10 may be vaulted, i.e., it may bridge over the exposed anterior surface of the primarily intraocular lens. Thus, the piggy-back lens 10 may not be supported by the anterior surface of the IOL 110. Therefore, it may be desirable that the piggy-back lens 10 be constructed from a material that provides structural support for the lens body 12, such that the lens body 12 is sufficiently rigid or stiff. The stiff lens body 12 may ensure that the piggy back lens 10 maintains its shape over time so as to provide the desired optical correction.

Therefore, embodiments of the present invention provide improved treatment for residual refractive error because the underlying pseudophakic refractive error is already known and stable so that the additive refractive treatment provided by the piggy-back lens 10 is very predictable. Inducement of astigmatism from surgery is a problem in predicting the final result which will be avoided because the piggy-back lens can be implanted through the original incision which has already induced astigmatism. Thus, the piggy-back lens 10 can be used to correct astigmatism (e.g., by having different optical powers in more than one meridians) created from the original IOL placement without inducing additional astigmatism.

A piggy-back lens 10 according to one aspect of the invention may include one or more of the following features: silicone material for the lens body to avoid intralenticular opacification (ILO); 3-piece ciliary sulcus fixation to prevent IOL rotation and provide stable astigmatism correction; concave-convex shape to minimize iris trauma, avoid pigment dispersion syndrome/glaucoma, and/or to prevent rotation; an optic diameter of about 7.0-mm or greater (D1 in FIG. 2) to cover the primary IOL 110 and to treat pseudophakic dysphotopsia (e.g., unwanted images after cataract surgery); an outer diameter of about 14.0-mm or greater (D2 in FIG. 2) for good ciliary sulcus fixation; PMMA haptics (or other stiff material) to maintain centration and to prevent rotation; cryolathable for custom order of perfect sphere, cylinder and even higher order aberrations and presbyopia correction.

In accordance with one aspect of the invention, the piggy-back lens 10 may be a concave/convex three piece intraocular lens that hugs the originally inserted IOL 110 and wraps around the IOL 110 with an optic diameter between about 7.0 and 8.0 mm. Because the desired correction provided by the piggy-back lens 10 is based on refraction, extremely accurate correction of astigmatism and other refractive complaints can be made with a minor surgery that may take only several minutes to perform (e.g., by implanting the piggy-back lens through the incision made for the original IOL 110). The piggy-back lens 10 does not involve ablating the corneal surface (such as is done in other procedures, e.g., LASIK surgery) which often leads to dry eye symptoms in the elderly who are those most likely to have had cataract surgery. In addition, LASIK is not as accurate and requires a large investment by the surgeon.

It will be appreciated that the present invention can be used in a variety of apparatuses and methods. For example, a piggy-back lens in accordance with the present invention may include a lens body having a convex anterior surface, a concave posterior surface, and a circumferential edge, wherein the circumferential edge has a rounded anterior portion and a rounded posterior portion; and at least two haptics extending from the lens body. The piggy-back lens may also include: a lens body comprised of silicone; a lens body having a refractive index equal to or less than about 1.48; a lens body having different optical powers in at least two different meridians to correct for astigmatism; a lens body may having a diameter of between about 7.0 to 8.0 mm; and/or haptics which anteriorly angled from the lens body at an angle of about 5 to 10 degrees; or combinations thereof.

In accordance with another aspect of the invention an intraocular lens may include a lens body and a haptic extending at an angle anteriorly from the lens body. The intraocular lens may also include a haptic that extends anteriorly from the lens body at an angle of about 5 to 10 degrees; an outer diameter of about 14 mm or greater; a haptic configured to contact the ciliary sulcus of an eye to secure the lens in a relatively fixed position in the eye; a haptic formed separately from the lens body and configured to be attached to the lens body; and/or a lens body has a diameter of between about 7.0 to 8.0 mm, or combinations thereof.

A method of correcting residual error in an eye after implantation of an intraocular lens in the eye may include the steps of: inserting a piggy-back lens into the eye, the piggy-back lens including a lens body and at least two haptics extending from the lens body, the lens body having a convex anterior surface, a concave posterior surface, and a circumferential edge; positioning the lens body in the eye so that the posterior surface of the lens body lies generally flush with an anterior surface of the intraocular lens with at least a portion of the posterior surface of the lens body contacting the anterior surface of the intraocular lens; and contacting outer portions of the at least two haptics with the ciliary sulcus of the eye to fix the piggy-back lens in the eye. The method may also include: at least about 25% of the posterior surface of the lens body contacting the anterior surface of the intraocular lens; at least about 50% of the posterior surface of the lens body contacts the anterior surface of the intraocular lens; at least about 75% of the posterior surface of the lens body contacting the anterior surface of the intraocular lens; a lens body which extends beyond a circumferential edge of the intraocular lens; a circumferential edge of the lens body which being rounded; a lens body comprised of silicone; a lens body having a refractive index equal to or less than about 1.48; the posterior surface of the lens body contacting a residual aspect of the anterior capsule, and the lens body substantially bridging over the exposed anterior surface of the intraocular lens; and/or inserting the piggy-back lens into the eye through an incision used to insert the intraocular lens into the eye; or combinations thereof.

An intraocular lens made in accordance with one aspect of the invention may include: a lens body and at least one haptic extending at an angle anteriorly from the lens body. The intraocular lens may also include: the at least one haptic extending anteriorly from the lens body at an angle of about 5 to 10 degrees; the lens body further having a flange for receiving the haptic, and the at least one haptic being is attached to the flange; wherein the at least one haptic is no greater than 100 microns in AP thickness and wherein the haptic is staked to the flange; wherein the at least one haptic is configured to contact the ciliary sulcus of an eye to secure the lens in a relatively fixed position in the eye; and/or the at least one haptic is formed separately from the lens body.

There may be many other ways to implement the invention. Various functions and elements described herein may be partitioned differently from those shown without departing from the spirit and scope of the invention. Various modifications to these embodiments will be readily apparent to those skilled in the art, and generic principles defined herein may be applied to other embodiments. Thus, many changes and modifications may be made to the invention, by one having ordinary skill in the art, without departing from the spirit and scope of the invention.

A reference to an element in the singular is not intended to mean “one and only one” unless specifically stated, but rather “one or more.” The term “some” refers to one or more. Underlined and/or italicized headings and subheadings are used for convenience only, do not limit the invention, and are not referred to in connection with the interpretation of the description of the invention. All structural and functional equivalents to the elements of the various embodiments described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

1-21. (canceled)

22. A piggy-back lens configured to be positioned anterior to a primary intraocular lens and outside the capsular bag, comprising:
a piggy back lens body having a convex anterior surface, a concave posterior surface, and a rounded circumferential edge, wherein the circumferential edge has a fully rounded anterior edge and a rounded posterior edge that form a smoothly rounded circumferential edge, wherein anterior and posterior is relative to the direction of the optical axis, wherein the lens body is hydrophobic, has a refractive index equal to or less than about 1.48, is configured to extend beyond the circumference of the primary intraocular lens, and has an optical diameter of about 7.0 or greater; and
at least two anteriorly angled haptics extending from the circumferential edge of the piggy back lens body, wherein the haptics have an overall outside diameter across the piggy-back lens of about 14 mm or greater.

23. The piggy-back lens of claim 22, wherein the lens body comprises silicone.

24. The piggy-back lens of claim 22, wherein the lens body has different optical powers in at least two different meridians to correct for astigmatism.

25. The piggy-back lens of claim 22, wherein the haptics are anteriorly angled from the lens body at an angle of about 5 to 10 degrees.

26. The piggy-back lens according to claim 22, wherein the lens body further comprises a flange for providing a secure attachment location for the at least one haptic, and wherein the at least one haptic is formed separate from the lens body and attached to the lens body via the flange.

27. The piggy-back lens according to claim 22, wherein the at least one haptic comprises a haptic which is no greater than 100 microns in anterior-posterior thickness.

28. The piggy back lens and primary intraocular lens combination of claim 22, wherein the smoothly rounded circumferential edge is semi-circular in shape.

29. The piggy back lens of claim 22, wherein the piggy back lens is implanted in the eye of a patient and further comprises a primary intraocular lens implanted in the eye of a patient, wherein the a piggy back lens body is positioned anterior to the primary intraocular lens and extends beyond the circumference of the primary intraocular lens; and wherein the at least two anteriorly angled haptics extending outwardly from the circumferential edge of the piggy back lens body push the piggy back lens body in a posterior direction such that the convex anterior surface of the piggy back lens body contacts at least a portion of the posterior surface of the primary intraocular lens.

30. A piggy-back lens for supplementing an intraocular lens implanted in an eye to correct for residual refractive error and/or other error, comprising:
a piggy-back lens body configured for contacting a primary intraocular lens, the lens body having a convex anterior surface, a concave posterior surface, and a fully rounded circumferential edge, wherein the piggy back lens body has a refractive index equal to or less than about 1.48, wherein the circumferential edge has a rounded anterior edge and a rounded posterior edge that form a smoothly rounded cirumferential edge, wherein anterior and posterior is relative to the direction of the optical axis, and an optical diameter of about 7.0 or greater;
at least two haptics extending from the circumferential edge of the piggy back lens body;
wherein the piggy back lens body is positionable such that the posterior surface of the piggy back lens body is configured to lie flush with an anterior surface of the primary intraocular lens with at least a portion of the posterior surface of the piggy back lens body configured to contact the anterior surface of the primary intraocular lens, and wherein the piggy back lens body is configured to extend beyond a circumferential edge of the primary intraocular lens.

31. The piggy-back lens of claim 30, wherein at least about 25% of the posterior surface of the lens body is configured to contact the anterior surface of the primary intraocular lens.

32. The piggy-back lens of claim 30, wherein at least about 50% of the posterior surface of the lens body is configured to contact the anterior surface of the primary intraocular lens.

33. The piggy-back lens of claim 30, wherein at least about 75% of the posterior surface of the lens body is configured to contact the anterior surface of the primary intraocular lens.

34. The piggy-back lens of claim 30, wherein the lens body comprises silicone.

35. The piggy-back lens of claim 30, wherein the at least two haptics are formed separately from the lens body, and wherein the lens body further comprises a flange for attaching the at least two haptics to the lens body.

36. The piggy back lens and primary intraocular lens combination of claim 35, wherein the smoothly rounded circumferential edge is semi-circular in shape.
